(19) United States
(12) Patent Application Publication
(10) Pub. No.: US 2017/0036986 A1
(43) Pub. Date: Feb. 9, 2017

(54) METHOD FOR EXTRACTING ACTIVE PRINCIPLES FROM ANTRODIA CAMPHORATA

(71) Applicant: MA SHEN KAI RUEI CO., LTD., Tainan City (TW)

(72) Inventor: Po-Liang Chen, Taipei City (TW)

(21) Appl. No.: 14/819,846

(22) Filed: Aug. 6, 2015

(51) Int. Cl. C07C 51/42; C07C 45/78

(52) U.S. Cl. CPC C07C 51/42; C07C 45/78

(57) ABSTRACT

A method for extracting active principles from fruiting bodies of Antrodia camphorata comprises the steps of: extracting fruiting bodies of Antrodia camphorata with hot water to obtain extracts HW, and thus extraction residues (residues HW) are remained; extracting the residues HW by a fractional distillation to obtain extracts FD, and thus extraction residues (residues FD) are remained; extracting the residues FD by immersing with a low polar solvent to obtain extracts LPS, and thus extraction residues (residues LPS) are remained; extracting the residues LPS through a cryo-condensation process to obtain extracts IEW, and thus extraction residues (residues IEW) are remained; extracting the residues IEW through a SCF by using CO₂ as a solvent to obtain extracts SCF, and thus extraction residues (residues SCF) are remained; and forming a mixture by mixing one or more extracts selected from the group consisting of extracts HW, FD, LPS, IEW, and SCF.

### BACKGROUND OF THE INVENTION

1. Field of the invention

The present invention relates to a method for extracting, particularly to a method for extracting active principles from fruiting bodies of Antrodia camphorata.

2. Descriptions of Related Art

Antrodia camphorata and the mycelia products therefrom or thereof possesses edible high value with various excellent functions not only in medicinal such as having anti-oxidant, antihypersensitive and immunostimulatory effects but also the capability of improving physical health by its own anticancer activity, reduced treatment-related symptoms and other side effects similar to medical efficiency of the wild fruiting bodies.

Consequently, many products made from Antrodia camphorata and/or comprising especially the active ingredient extracted form thereof such as Antrodia oil, Antrodia extraction, Antrodia combination and so on, are broadly used in various medicine, health care applications and also Antrodia camphorata and wild cattle camphor thus has been listed as one of the biological treasure in recent years by the Taiwan Government.

Antrodia camphorata is a non-mesh skirt bacteria, an endemic fungus, and grows in the internal heartwood (or the dark/humid wood surface) of Cattle camphorin the mountainous region of Taiwan, altitude 450-2000 meters mountain forest. It is also perennial mushroom fungus and only grows in the inner wall of a wood trunk decayed decades or more, or the lodging of the dead wood wet surface of a Cattle camphor, particularly Cinnamomum kanehirai.

Antrodia camphorata is rich in Triterpenoids, immunostimulatory polysaccharides such as—D-glucan polysaccharides, Adenosine, Nicotinic acid, SOD (superoxide dismutase enzymes), Steroids, Vitamin, essential minerals, and other pharmaceutically active principles.

In addition, Antrodia extraction and/or Antrodia oil also contains much important nutrients to the human body, for examples, oleic acid, palmitic acid, linoleic acid, palmitoleic acid, linolenic acid, stearic acid, meat, beans Qu acid, arachidic acid, behenic acid, tetracosanoic acid, n-heptadecyl acid, n-heptadecenoic acid, vitamin A, vitamin B, vitamin E and minerals; as well as it also can inhibit tumor metastasis, reduce the incidence of coronary heart disease, improve immunity, and other effects.

Thereby, Antrodia camphorata shows various excellent functions such as detoxification, hypoglycemic effects, reducing blood pressure, improving anti-cancer effect, inhibition of histamine release effect, enhancing anti-inflammatory effect; enhancing immunity, increasing cell viability, eliminating free radicals, promoting liver cell regeneration, lowering down alanine aminotransferase, and in addition to even enhancing the phagocytic capacity of macrophages as well as having the capability in improving physical health.

In general, these pharmaceutically active principles as above-mentioned are majorly presented in fruiting body or mycelia of Antrodia camphorata and can be extracted therefrom by conventional extraction methods of using various organic solvents, in organic solvents and/or water, as well as by means of several different extraction methods includes distillation, filtration, fractional distillation, supercritical fluid extraction (SFE), semi-bionic extraction, ultrasonic cycle technology, chemical derivation and others in the prior art.

Among them, in traditional extraction methods, the organic solvents used for extracting fruiting body or mycelia of Antrodia camphorata generally include ethanol and/or water through an extraction/separation process, however, it is too complicated in operation for obtaining pharmaceutically active principles such as polysaccharides having efficiency in inhibiting matrix metalloproteinase activities.

For example, Taiwan Patent No. 1299665 discloses an extract of Antrodia camphorata extracted from the mycelia of Antrodia camphorata by using ethanol. In addition, Taiwan Patent No. 591110 discloses a y-aminobutyric acid extracted from the lyophilized mycelia of Antrodia camphorata with water or organic solvents. However, it is not extracted from the fruiting body of Antrodia camphorata with water or organic solvent, and no component is identified, especially, either the extract or the mycelia product thereof is short of pharmaceutically active principles and it thus cannot inhibit cancer cell growth.

On the other hand, the supercritical fluid method (SFE) is often used for extracting fruiting body or mycelia of Antrodia camphorata in view of it contains a variety of fat compounds.

However, Antrodia camphorata cannot be artificially cultured, and further the wild fruiting body of Antrodia camphorata is very little and cannot be easily found out. As a result, Antrodia camphorata thus comes to be very expensive. Particularly, the conventional extraction method used in the prior arts still have several defects covering at least a higher cost, lower product yield and residual organic solvent which is effective ingredient in medicinal.

Thus, it is required a useful method for extracting an effective ingredient from Antrodia camphorata more than prior arts or making a novel combination, composition or mixture having excellent medicinal efficiency superior to prior arts. To this end, the present invention is an extract from Antrodia quick access to high levels of total triterpenoids, and there are no adverse effects on the environment and the whole operation the operator.

### SUMMARY OF THE INVENTION

An aspect of the present invention is to provide a method for extracting active principles from fruiting bodies of Antrodia camphorata, wherein said method comprises the following steps: (A) extracting fruiting bodies of Antrodia camphorata with hot water at a temperature in a range of 45° C. to 100° C. to obtain extracts HW, and thus extraction residues, referred to as residues HW, are remained; (B) extracting the residues HW by a fractional distillation to obtain extracts FD which are collected from a condensation liquid in a fractional distillation apparatus, and thus extraction residues, referred to as residues FD, are remained; (C) extracting the residues FD by immersing with a low polar solvent at least for 4 hours to obtain extracts LPS, and thus extraction residues, referred to as residues LPS, are remained; (D) extracting the residues LPS through a cryo-condensation process by dropping an iced ethanol/water with a temperature in a range of 0° C. to 15° C. to obtain extracts IEW, and thus extraction residues, referred to as residues IEW, are remained; (E) extracting the residues IEW through a SCF (supercritical fluid extraction) by using CO₂ as a solvent at a temperature of 31.26° C. and a pressure of 72 atm to obtain extracts SCF, and thus extraction residues, referred to as residues SCF, are remained, and (F) forming a mixture by mixing one or more extracts selected from the group consisting of extracts HW, extracts FD, extracts LPS, extracts IEW, and extracts SCF.

According to the present invention, an extracting method capable of selected from a group consisting of hot water extraction, distillation, supercritical fluid extraction, cryo-condensation, and ethanol infusion.

According to the present invention, a useful method for extracting Antrodia camphorata capable of acquiring active principles with diverse polarities more than prior arts is provided.

According to the present invention, a mixture comprising at least one or more selected from a group consisting of antroquinonol, antrocinnamonin A, antroquinonol B, antroquinonol D, dehydroeburicoic acid, dehydrosulphurenic acid, zhankuic acid A, zhankuic acid C, antcin K, and antcin C is provided, especially, the component and the blending ratio thereof in the mixture can be altered to obtain specific ratio of the active principles.

Another one aspect of the present invention is to provide a method for extracting active principles from fruiting bodies of Antrodia camphorata, wherein the fruiting bodies of Antrodia camphorata are artificially-fermented or gathered from wild Taiwanofungus camphoratus.

The other one aspect of the present invention is to provide a method for extracting active principles from fruiting bodies of Antrodia camphorata, wherein one of extracts HW, extracts FD, extracts LPS, extracts IEW, and extracts SCF comprises at least an active principles selected from a group consisting of antroquinonol, antrocinnamonin A, antroquinonol B, antroquinonol D, dehydroeburicoic acid, dehydrosulphurenic acid, zhankuic acid A, zhankuic acid C, antcin K, and antcin C.

In addition, one aspect of the present invention is to provide a method for extracting active principles from fruiting bodies of Antrodia camphorata, wherein the hot water used in the step (A) has a temperature in the range of 55 to 85° C.

Besides, another one aspect of the present invention is to provide a method for extracting active principles from fruiting bodies of Antrodia camphorata, wherein the low polar solvent used in the step (C) is selected from the group consisting of water, ethanol, ethanol/water mixture, methanol, butanol, n-butanol, isobutanol, acetone, hexane, n-hexane, petroleum ether, ethyl acetate, methylene chloride, and trichloro methane.

The other one aspect of the present invention is to provide a method for extracting active principles from fruiting bodies of Antrodia camphorata, wherein the cryo-condensation processes of step (D) is proceeded at the temperature in a range of 2 to 10° C.

Additionally, one aspect of the present invention is to provide a method for extracting active principles from fruiting bodies of Antrodia camphorata, wherein a solvent is further added in the supercritical fluid extraction of the step (E), and the solvent is at least the on selected from the group consisting of an organic solvent, an inorganic solvent, a low polar solvent, a high polar solvent, a water, and a combination thereof.

Further, one aspect of the present invention is to provide a method for extracting active principles from fruiting bodies of Antrodia camphorata, wherein the low polar solvent further used in the supercritical fluid extraction of the step (E), comprises at least one selected from the group consisting of water, ethanol, ethanol/water mixture, methanol, butanol, n-butanol, isobutanol, acetone, hexane, n-hexane, petroleum ether, ethyl acetate, methylene chloride, and trichloro methane.

Another one aspect of the present invention is to provide a method for extracting active principles from fruiting bodies of Antrodia camphorata, wherein in the step (F), the mixture consists of 15 to 25% of extracts HW, 15 to 25% of extracts FD , 15 to 25% of extracts LPS , 15 to 25% of extracts IEW, and 15 to 25% of extracts SCF.

The other one aspect of the present invention is to provide a method for extracting active principles from fruiting bodies of Antrodia camphorata, wherein in the step (F), further included is processing the mixture by fractional distillation to obtain a purified mixture comprising one or more extracts selected from the group consisting of extracts HW, extracts FD, extracts LPS, extracts IEW, and extracts SCF.

One aspect of the present invention is to provide a method for extracting active principles from fruiting bodies of Antrodia camphorata, wherein in the step (F), further comprises granulating the mixture into a shaped article by adding at least one excipient or vehicle selected from the group consisting of diluents, binders, compression aids, granulating agents, disintegrants, glidants, lubricants, tablet coatings, tablet films, and coloring agents.

Further, another one aspect of the present invention is to provide a method for extracting active principles from fruiting bodies of Antrodia camphorata, wherein the excipient or vehicle is selected from the group consisting of silica, silicon compound, colloidal anhydrous silicon, stearic acid, magnesium stearate, sugar, sucrose, an acid-insoluble polymer, cellulose acetate phthalate, synthetic dyes, natural colours, natural pigments, and phospholipids.

Another one aspect of the present invention is to provide a method for extracting active principles from fruiting bodies of Antrodia camphorata, wherein the shaped article further includes a liposome structure formed from at least a phospholipid in interior.

The other one aspect of the present invention is to provide a method for extracting active principles from fruiting bodies of Antrodia camphorata, wherein the phospholipid is at least one selected from a group consisting of phosphatidylcholine, phosphatidylserine, and phosphatidylinositol.

Further, according to the useful method for extracting Antrodia camphorata of the present invention, an extract extracted from Antrodia camphorata, having effective ingredients and also physical/chemical properties superior to prior arts is provided.

Besides, according to the useful method for extracting Antrodia camphorata of the present invention, a novel combination, composition or mixture having excellent medicinal efficiency superior to prior arts is further provided.

In addition, according to the useful method for extracting Antrodia camphorata of the present invention, an extract having high levels of total triterpenoids is provided.

According to the present invention, an useful method for extracting Antrodia camphorata without adverse effects on the environment but easy operation to the operator and industry is provided.

Additionally, according to the present invention, a shaped article comprising the active principles selected from a group consisting of antroquinonol, antrocinnamonin A, antroquinonol B, antroquinonol D, dehydroeburicoic acid, dehydrosulphurenic acid, zhankuic acid A, zhankuic acid C, antcin K, and antcin C is provided, especially, a shaped article with a liposome structure capable of making the active principles more absorbable can be provided by adding phospholipids in the mixture of extracts.

### BRIEF DESCRIPTION OF DRAWINGS

FIG. 1 illustrates a typical scheme of flowchart of a method for extracting active principles from fruiting bodies of Antrodia camphorata according to the invention.

### DETAILED DESCRIPTION

The present invention will now be described more specifically with reference to the following embodiments. It is to be noted that the following descriptions of preferred embodiments of this invention are presented herein for purpose of illustration and description only; it is not intended to be exhaustive or to be limited to the precise form disclosed.

Unless defined otherwise herein, or scientific and technical terms used herein shall have the ordinary skill in the art commonly understood meaning The meaning and scope of those terms should be clear; however, in the case of any potential ambiguity, definitions provided herein are better than any dictionary or extrinsic definition.

Unless otherwise indicated, the following terms as used in this disclosure should be understood to have the following meanings.

As used herein, the term “cancer” refers to the loss of control of cell growth of lung tissue diseases. This may result in the transfer of cell growth, that is, invasion of adjacent tissue and infiltration beyond the lungs.

As used herein, the term “prevention” refers to an individual suffering from a disease symptom delay the onset or reduce disease appear.

As used herein, the term “treating” means to alleviate or ameliorate symptoms of susceptible individuals.

As used herein, the term “therapeutically effective amount” means alone or in combination with other therapeutic/pharmaceutical compositions for the treatment of lung cancer using amount display therapeutic efficacy of the active ingredient.

The term “carrier” or “pharmaceutically acceptable carrier” means a person of ordinary skill in the art is well known for the preparation of pharmaceutical compositions diluents, excipients, acceptor (receptor) or like.

As used herein, the term “subject” means animals, especially mammals In a preferred embodiment, the term “individual” means “humanity.”

As used herein, the term “extract” is meant a substance for extraction of the resulting product, usually extracted by soaking Suoyu substance or mixed in a solvent to obtain a solution or concentrated formulation. Typically, the system extracts prepared from fresh or dried plants or plant milled samples.

Unless the context requires otherwise, the singular terms shall include the plural and plural terms shall include the singular.

The present invention provides a variety of method for extracting active principles as Antrodia extract from fruiting bodies of Antrodia camphorata.

### (Pre-Treatment of Fruiting Bodies or Mycelia of Antrodia Camphorata)

Even though the fruiting bodies is commonly deemed as having better medical efficacy than that of mycelia, however please be noted either fruiting bodies or mycelia of Antrodia camphorata can be selected on basis of situation and requirement according to one aspect of the present invention. According to one aspect of the present invention, the fruiting bodies of Antrodia camphorata may be artificially-fermented or gathered from wild Taiwanofungus camphoratus. However, a wild Taiwanofungus camphoratus is more preferable.

Generally, as to Antrodia camphorata, the ratios of active principles are various in fruiting bodies and mycelia. However, there is much impurities such as dust, slime or others presented in either the surface or crack space in the interior of the fresh fruiting bodies and mycelia of Antrodia camphorata, and thereby it is preferred to be pre-treated prior to be extracted

According to the present invention, the fruiting bodies of Antrodia camphorata gathered from wild Antrodia camphorata or artificially-fermented Antrodia camphorata is preferably to be pre-treated. For example, the fresh fruiting bodies of Antrodia camphorata may firstly be cleaned and cut appropriately into small pieces or slices to be a sample (FBS) with an appropriate size.

Further, the FBS sample was put into drying in an oven with a constant temperature in a range of 45° C. to 50° C. under ventilation for 16 to 24 hrs and thereby a dry sample (DFBS). If required, the dried DFBS sample may further be processed into flour by a mill and then Antrodia camphorata fruiting body powder (PFB) was thus obtained.

The weight of the DFBS sample was scaled after being dried for 16 to 24 hrs, and a dry weight ratio % was calculate by a formula as follows:

dry weight ratio %=(dry sample weight/wet sample weight)*100%

The calculation results of dry weight ratio of DFBS samples are individually shown in Table 1 as below:

| Batch No. | Dry Weight Ratio (%) |
|---|---|
| 1 | 36.8 |
| 2 | 37.2 |
| 3 | 35.9 |
| 4 | 36.3 |
| 5 | 37.7 |

Further, please be advised while conducting a pre-treatment, the fruiting bodies of Antrodia camphorata has better to be cleaned in the beginning and may merely to be cut into small pieces or slices without being milled to powder, but it is not limited to do so. Besides, in one aspect of the present invention, any processing procedure for pre-treatment may be omitted.

After pre-treatment, the DFBS (fruiting bodies of Antrodia camphorata) are further to subject to processing by any suitable extracting methods to obtain extracts. A variety of extraction methods is known in the art, however, the extracting method includes, but is not limited to, for example, it may be dipping, percolation, and then percolation, digestion, countercurrent extraction, turbine extraction, extrusion/pressing/pressing or supercritical fluid carbon dioxide extraction.

### (Extracting Method for Antrodia Camphorata)

Generally, in the present invention, the extracting method is not limited, however preferably comprises at least one selected from a group consisting of hot water extraction, distillation, fractional distillation, supercritical fluid extraction, cryo-condensation, and ethanol infusion and other methods used in the prior arts in this field, as well as a combination thereof.

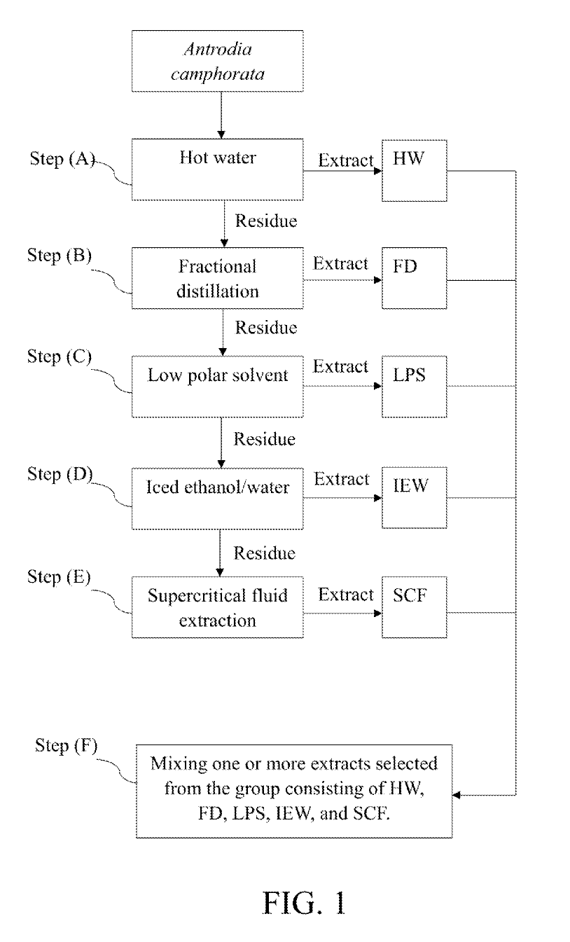

According to one aspect of the present invention, a method for extracting active principles from fruiting bodies of Antrodia camphorata is provided. A typical scheme flowchart of a method for extracting active principles from fruiting bodies of Antrodia camphorata, according to the invention, is shown in FIG. 1. As the flowchart shown in FIG. 1, the method according to the present invention, it comprises the following step (A) of hot water extraction, step (B) of fractional distillation extraction, step (C) of low polar solvent extraction, step (D) of iced ethanol/water extraction, step (E) of supercritical flow extraction, and step (F) of mixture of extractions.

Specifically, the method according to the invention comprises the following steps: (A) extracting fruiting bodies of Antrodia camphorata with hot water at a temperature in a range of 45° C. to 100° C. to obtain extracts HW, and thus extraction residues, referred to as residues HW, are remained; (B) extracting the residues HW by a fractional distillation to obtain extracts FD which are collected from a condensation liquid in a fractional distillation apparatus, and thus extraction residues, referred to as residues FD, are remained; (C) extracting the residues FD by immersing with a low polar solvent at least for 4 hours to obtain extracts LPS, and thus extraction residues, referred to as residues LPS, are remained; (D) extracting the residues LPS through a cryo-condensation process by dropping an iced ethanol/water with a temperature in a range of 0° C. to 15° C. to obtain extracts IEW, and thus extraction residues, referred to as residues IEW, are remained; (E) extracting the residues IEW through a SCF (supercritical fluid extraction) by using CO₂ as a solvent at a temperature of 31.26° C. and a pressure of 72 atm to obtain extracts SCF, and thus extraction residues, referred to as residues SCF, are remained, and (F) forming a mixture by mixing one or more extracts selected from the group consisting of extracts HW, extracts FD, extracts LPS, extracts IEW, and extracts SCE

### (Step (A) of the Extracting Method for Antrodia Camphorata)

According to the present invention, in the step (A), hot water extraction is proceed, Namely, the fruiting bodies of Antrodia camphorata is extracted to obtain extracts HW by using a hot water at a temperature in a range of 45° C. to 100° C. Accordingly, an extract comprises at least an active principles selected from a group consisting of antrocinnamonin A, antroquinonol B, dehydroeburicoic acid, dehydrosulphurenic acid, zhankuic acid A, zhankuic acid C, antcin K, and antcin C can be obtained in the step (A).

The hot water extraction of the present invention is proceeded by using a water at a temperature in the range of 45 to 100° C. used; preferably use a hot water having at temperature in a range of 55° C. to 85° C.; more preferably use a hot water at temperature in a range of 55° C. to 70° C.

Besides, according to the invention, a hot water is also used as an illustrated example of getting extracts as well as simultaneously removing the bitter taste of the fruiting bodies of Antrodia camphorata.

### (Step (B) of the Extracting Method for Antrodia Camphorata)

According to the present invention, in the step (B), a distillation or fractional distillation may be used to obtain extracts, which can be collected from a condensation liquid. Besides, the extract obtained by the step (B) of the invention comprises at least an active principles selected from a group consisting of antroquinonol, dehydroeburicoic acid, dehydrosulphurenic acid, zhankuic acid A, zhankuic acid C, antcin K, and antcin C can be obtained.

For example, the small pieces or slices of the fruiting bodies of Antrodia camphorata such as the residues of (A) are put into a distillation device or a fractional distillation apparatus, then an extraction is proceed and further the condensate is gathered by means of a fractional distillation.

According to one aspect of the present invention, the temperature in the distillation process may be at a range of about 5° C. to about 100° C., preferably at a range of about 10° C. to about 100° C., more preferably at a range of about 20° C. to about 100° C., further more preferably at a range of about 40° C. to about 100° C., and most preferably at a range of about 60° C. to about 100° C., preferably at room temperature 25° C. or 100° C. boiling.

### (Step (C) of the Extracting Method for Antrodia Camphorata)

According to the present invention, in the step (C), extraction with a solvent is proceeded to the residues of the step (B) or the fruiting bodies of Antrodia Camphorata by immersing with at least for 4 hours to obtain extracts, which comprises at least an active principles selected from a group consisting of antroquinonol, antrocinnamonin A, antroquinonol B, antroquinonol D, dehydroeburicoic acid, dehydrosulphurenic acid, zhankuic acid A, zhankuic acid C, antcin K, and antcin C can be obtained.

According to one aspect of the invention, the solvent is at least the on selected from the group consisting of an organic solvent, an inorganic solvent, a low polar solvent, a high polar solvent, a water, and a combination thereof.

According to one aspect of the present invention, a suitable solvents include but is not limited to, water, ethanol, ethanol/water mixture, methanol, butanol, n-butanol, isobutanol, acetone, hexane, n-hexane, petroleum ether, ethyl acetate, methylene chloride, trichloro methane or other solvents. For example, low polarity solvents may preferably be selected from the group consisting of petroleum ether, hexane, methylene chloride, chloroform, ethyl acetate, acetone, and ethanol. The highly polar solvents may preferably be selected from the group consisting of water, ethanol, methanol, butanol, isobutanol, and acetone.

According to the present invention, the low polar solvent used in the step (C) is selected from the group consisting of water, ethanol, ethanol/water mixture, methanol, butanol, n-butanol, isobutanol, acetone, hexane, n-hexane, petroleum ether, ethyl acetate, methylene chloride, and trichloro methane.

According to an aspect of the invention, the concentration of solvent is not limited and can be selected by requirement. For example, as to low polarity solvents such as petroleum ether, hexane, methylene chloride, chloroform, ethyl acetate, acetone, and ethanol and so on, it may use in a rage of 50-100%, preferably in a rage of 70-100%. As to highly polar solvents such as water, ethanol, methanol, butanol, isobutanol, acetone and so on, it may use in a rage of less than 80%, preferably in a rage of 50% or less for a solvent other than acetone.

According to an aspect of the invention, the ratio of solvent with respect to extractable materials is not limited and can be selected by requirement. For example, according to one aspect of the present invention, it can be from about 1:1 to about 1:100 (g/ml), preferably about 1:1 to about 1:50 (g/ml), more preferably from about 1:1 to about 1:0.20, (g/ml), and most preferably about 1:15 or 1:10 (g/ml).

According to an aspect of the invention, an ethanol infusion process is preferably used, for example, the small pieces or slices of the fruiting bodies of Antrodia camphorata are put into ethanol (over 25%), soaked several hours to years, and then filtered.

Additionally, in order to achieve a suitable polar extraction, according to an aspect of the invention, the required concentration of solvent can be selected according to the type of agent or formulation the solvent. The suitable solvent to be used in the present invention includes, but is not limited to low polarity solvents or highly polar solvents.

### (Step (D) of the Extracting Method for Antrodia Camphorata)

According to the invention, in the step (D), the residues of the step (C) may further extracted through a cryo-condensation process by dropping an iced ethanol/water with a temperature in a range of 0° C. to 15° C. to obtain extracts, which comprises at least an active principles selected from a group consisting of antroquinonol, antrocinnamonin A, antroquinonol B, antroquinonol D, dehydroeburicoic acid, dehydrosulphurenic acid, zhankuic acid A, zhankuic acid C, antcin K, and antcin C.

According to the method for extracting active principles from fruiting bodies of Antrodia camphorata of the present invention, the cryo-condensation, ice and ethanol may be mixed and dropped into the small pieces or slices of the fruiting bodies of Antrodia camphorata. Then the condensate from ice drip process is harvested and filtered. Further, the cryo-condensation processes may proceeded at the temperature in a range of 0° C. to 15° C.; preferably is proceeded at the temperature in a range of 2° C. to 10° C.; more preferably is proceeded at the temperature in a range of 2° C. to 8° C.

### (Step (E) of the Extracting Method for Antrodia Camphorata)

According to the invention, in the step (E), the residues of the step (D) may further extracted through a SCF (supercritical fluid extraction) by using CO₂ as a solvent at a temperature of 31.26° C. and a pressure of 72 atm to obtain extracts, which comprises at least an active principles selected from a group consisting of antroquinonol, antrocinnamonin A, antroquinonol B, antroquinonol D, dehydroeburicoic acid, dehydrosulphurenic acid, zhankuic acid A, zhankuic acid C, antcin K, and antcin C.

The supercritical fluid has both the characteristics of the liquid dissolving capacity have gas diffusion and features easy motion, the mass transfer rate is much higher than the liquid phase process. Solubility and density of the supercritical fluid has a close relationship.

At a certain temperature, the solubility of the supercritical fluid increases with increasing pressure, when the pressure is increased to a certain extent, will be out of inversion region, then the solubility with increasing temperature increases. Especially in small changes near the critical point temperature or pressure can cause solubility mutation in several orders of magnitude.

Namely, contact pressure and temperature control system of the supercritical fluid makes it the selective extraction of some of these components, and then adjust the temperature and pressure changes, reducing the density of the supercritical fluid, to achieve the separation of the extracted material.

Thus, the supercritical fluid method (SFE) is often used for extracting fruiting body or mycelia of Antrodia camphorata in view of it contains a variety of fat compounds.

According to the invention, in the supercritical fluid extraction, the small pieces or slices of the fruiting bodies of Antrodia camphorata may be treated with CO₂ at a low temperature and under high pressure to extract the triterpenoids and lipophilic compounds out from the fruiting bodies of Antrodia camphorata.

Besides, according to one aspect of the present invention, a solvent is further added in the supercritical fluid extraction of the step (E), and the solvent is at least the on selected from the group consisting of an organic solvent, an inorganic solvent, a low polar solvent, a high polar solvent, a water, and a combination thereof.

Further, one aspect of the present invention, a low polar solvent further used in the supercritical fluid extraction of the step (E), comprises at least one selected from the group consisting of water, ethanol, ethanol/water mixture, methanol, butanol, n-butanol, isobutanol, acetone, hexane, n-hexane, petroleum ether, ethyl acetate, methylene chloride, and trichloro methane.

### (Step (F) of the Extracting Method for Antrodia Camphorata)

According to the invention, in the step (F), the extracts HW, extracts FD, extracts LPS, extracts IEW, and extracts SCF in the step(A) to step (E) may further mixed to form a mixture of extracts, which comprises at least an active principles selected from a group consisting of antroquinonol, antrocinnamonin A, antroquinonol B, antroquinonol D, dehydroeburicoic acid, dehydrosulphurenic acid, zhankuic acid A, zhankuic acid C, antcin K, and antcin C.

According to another one aspect of the present invention, in the step (F), the mixture consists of 15 to 25% of extracts HW, 15 to 25% of extracts FD, 15 to 25% of extracts LPS , 15 to 25% of extracts IEW, and 15 to 25% of extracts SCF.

According to the other one aspect of the present invention, in the step (F), it further comprises processing the mixture by fractional distillation to obtain a purified mixture comprising one or more extracts selected from the group consisting of extracts HW, extracts FD, extracts LPS, extracts IEW, and extracts SCF.

Besides, it may further comprise granulating the mixture into a shaped article by adding at least one excipient or vehicle selected from the group consisting of diluents, binders, compression aids, granulating agents, disintegrants, glidants, lubricants, tablet coatings, tablet films, and coloring agents.

Further, according to another one aspect of the present invention, the excipient or vehicle is selected from the group consisting of silica, silicon compound, colloidal anhydrous silicon, stearic acid, magnesium stearate, sugar, sucrose, an acid-insoluble polymer, cellulose acetate phthalate, synthetic dyes, natural colors, natural pigments, and phospholipids.

According to another one aspect of the present invention, the shaped article further includes a liposome structure formed from at least a phospholipid in interior.

According to another one aspect of the present invention, the phospholipid is at least one selected from a group consisting of phosphatidylcholine, phosphatidylserine, and phosphatidylinositol.

Besides, extracts obtained at the different stages can be combined with each other, and also can be further concentrated for example, in the subsequent concentration step by means of such as evaporation or purification, or in separation steps by means of such as filtration, centrifugation, or chromatography.

### EXAMPLE

### (Extraction for Fruiting Body of Wild Antrodia Camphorata)

One (1 g) of the wild fruiting body of Antrodia camphorata after pre-treatment of cleaning and so on, was prepared to sliced into pieces. The prepared fruiting bodies of Antrodia camphorata was put into a hot water at a temperature in a range of 45° C. to 100° C. to for 2 hours. The extract named as extracts HW were obtained and the residues HW were remained.

The residues HW were then further extracted through a fractional distillation in a fractional distillation apparatus for 6 to 8 hours. The extracts FD were collected from a condensation liquid and extraction residues, referred to as residues FD, were remained.

Then, the residues FD were immersed with a low polar solvent of ethanol (25%) at least for 4 hours to 6months. The extracts LPS were obtained and thus residues LPS are remained.

The residues LPS were further extracted through a cryo-condensation process by dropping an iced ethanol/water with a temperature in a range of 0° C. to 15° C. for 3 to 6 hours. The extracts IEW were obtained and residues, referred to as residues IEW, were remained;

After then, the residues IEW is further extracted through a SCF (supercritical fluid extraction) by using CO₂ as a solvent at a temperature of 31.26° C. and a pressure of 72 atm. The extracts SCF were obtained and residues, referred to as residues SCF, are remained.

A mixture formed from extracts HW, extracts FD, extracts LPS, extracts IEW, and extracts SCF was obtained by mixing. If required, the supernatants were filtered and then centrifuged at 3,000 rpm for 30 minutes to remove the precipitate.

Further, the biologically active components of extracts HW, extracts FD, extracts LPS, extracts IEW, and extracts SCF were individually identified. Fifty-percent inhibition concentration (IC50) of extracts HW, extracts FD, extracts LPS, extracts IEW, and extracts SCF was determined as 104.82 μg/ml by MTT (3-(4,5-dimethylthiazol-2-yl)-2,5-diphenyltetrazolium bromide) assay; however, WEAC was inactive (IC50>200 μg/ml). Extracts HW, extracts FD, extracts LPS, extracts IEW, and extracts SCF (203.1 mg) was extracted with solvents of increasing polarities (n-hexane, ethyl acetate and ethanol sequentially), and three different extracts, 12.8% (26.0 mg) of n-hexane extract (FC), 61.6% (125.1 mg) of ethyl acetate extract (FA) and 8.4% (17.0 mg) of ethanol extract (FB), and 10.0% (20.2 mg) of the insoluble residue were yielded. Percentage is referred to yields in weight percentage of EEAC, milligram is referred to the corresponding dried weight after fractionation.

It will be apparent to those skilled in the art that various modifications and variations can be made to the present disclosure without departing from the spirit and scope of the disclosure. Thus it is intended that the present disclosure cover the modifications and variations of this disclosure provided they come within the scope of the appended claims and their equivalents.

What is claimed is:

1. A method for extracting active principles from fruiting bodies of Antrodia camphorata, wherein said method comprises the following steps:
(A) extracting fruiting bodies of Antrodia camphorata with hot water at a temperature in a range of 45° C. to 100° C. to obtain extracts HW, and thus extraction residues, referred to as residues HW, are remained;
(B) extracting the residues HW by a fractional distillation to obtain extracts FD which are collected from a condensation liquid in a fractional distillation apparatus, and thus extraction residues, referred to as residues FD, are remained;
(C) extracting the residues FD by immersing with a low polar solvent at least for 4 hours to obtain extracts LPS, and thus extraction residues, referred to as residues LPS, are remained;
(D) extracting the residues LPS through a cryo-condensation process by dropping an iced ethanol/water with a temperature in a range of 0° C. to 15° C. to obtain extracts IEW, and thus extraction residues, referred to as residues IEW, are remained;
(E) extracting the residues IEW through a SCF (supercritical fluid extraction) by using CO₂ as a solvent at a temperature of 31.26° C. and a pressure of 72 atm to obtain extracts SCF, and thus extraction residues, referred to as residues SCF, are remained, and
(F) forming a mixture by mixing one or more extracts selected from the group consisting of extracts HW, extracts FD, extracts LPS, extracts IEW, and extracts SCF.

2. The method according to claim 1, wherein the fruiting bodies of Antrodia camphorata are artificially-fermented or gathered from wild Taiwanofungus camphoratus.

3. The method according to claim 1, wherein one of extracts HW, extracts FD, extracts LPS, extracts IEW, and extracts SCF comprises at least an active principles selected from a group consisting of antroquinonol, antrocinnamonin A, antroquinonol B, antroquinonol D, dehydroeburicoic acid, dehydrosulphurenic acid, zhankuic acid A, zhankuic acid C, antcin K, and antcin C.

4. The method according to claim 1, wherein the hot water used in the step (A) has a temperature in the range of 55 to 85° C.

5. The method according to claim 1, wherein the low polar solvent used in the step (C) is selected from the group consisting of water, ethanol, ethanol/water mixture, methanol, butanol, n-butanol, isobutanol, acetone, hexane, n-hexane, petroleum ether, ethyl acetate, methylene chloride, and trichloro methane.

6. The method according to claim 4, wherein the cryo-condensation processes of step (D) is proceeded at the temperature in a range of 2 to 10° C.

7. The method according to claim 1, wherein a solvent is further added in the supercritical fluid extraction of the step (E), and the solvent is at least the on selected from the group consisting of an organic solvent, an inorganic solvent, a low polar solvent, a high polar solvent, a water, and a combination thereof.

8. The method according to claim 1, wherein the low polar solvent further used in the supercritical fluid extraction of the step (E), comprises at least one selected from the group consisting of water, ethanol, ethanol/water mixture, methanol, butanol, n-butanol, isobutanol, acetone, hexane, n-hexane, petroleum ether, ethyl acetate, methylene chloride, and trichloro methane.

9. The method according to claim 4, wherein in the step (F), the mixture consists of 15 to 25% of extracts HW, 15 to 25% of extracts FD, 15 to 25% of extracts LPS, 15 to 25% of extracts IEW, and 15 to 25% of extracts SCF.

10. The method according to claim 1, wherein in the step (F), further comprises processing the mixture by fractional distillation to obtain a purified mixture comprising one or more extracts selected from the group consisting of extracts HW, extracts FD, extracts LPS, extracts IEW, and extracts SCF.

11. The method according to claim 1, wherein in the step (F), further comprises granulating the mixture into a shaped article by adding at least one excipient or vehicle selected from the group consisting of diluents, binders, compression aids, granulating agents, disintegrants, glidants, lubricants, tablet coatings, tablet films, and coloring agents.

12. The method according to claim 11, wherein the excipient or vehicle is selected from the group consisting of silica, silicon compound, colloidal anhydrous silicon, stearic acid, magnesium stearate, sugar, sucrose, an acid-insoluble polymer, cellulose acetate phthalate, synthetic dyes, natural colours, natural pigments, and phospholipids.

13. The method according to claim 12, wherein the phospholipid is at least one selected from a group consisting of phosphatidylcholine, phosphatidylserine, and phosphatidylinositol.

14. The method according to claim 11, wherein the shaped article further includes a liposome structure formed from at least a phospholipid in interior.

15. The method according to claim 14, wherein the phospholipid is at least one selected from a group consisting of phosphatidylcholine, phosphatidylserine, and phosphatidylinositol.
